(19) United States
(12) Patent Application Publication
(10) Pub. No.: US 2023/0310709 A1
(43) Pub. Date: Oct. 5, 2023

(54) RETINAL GRAFT AND METHOD OF PREPARATION

(71) Applicant: Precise-Bio 3D LTD., Modiin Maccabim Reut (IL)

(72) Inventors: Amos EITAN, Jerusalem (IL); Aryeh Batt, Har Hebron (IL); Ariel Eisenbach, Zufim (IL); Dorin Sade Yazdi, Tel Aviv (IL); Michal Marcus, Kochav Yair (IL); Lior Rosenberg Belmaker, Shoham (IL); Maayan Malki, Petah Tikva (IL); Gal Shenbach-Koltin, Modiin Maccabim Reut (IL)

(21) Appl. No.: 18/129,875

(22) Filed: Apr. 2, 2023

Related U.S. Application Data
(63) Continuation-in-part of application No. 18/126,455, filed on Mar. 26, 2023.
Application No. 18/126,455 is a continuation of application No. 17/198,449, filed on Mar. 11, 2021.
(60) Provisional application No. 63/326,991, filed on Apr. 4, 2022.
(60) Provisional application No. 63/435,992, filed on Dec. 29, 2022.

(51) Int. Cl. A61L 27/36; A61L 27/24; A61L 27/38; B41J 3/407

(52) U.S. Cl. CPC A61L 27/3666; A61L 27/3604; A61L 27/24; A61L 27/3834; A61L 27/3869; A61L 27/3687; B41J 3/407; A61L 2430/16

(57) ABSTRACT

A method of manufacturing a retinal graft. The method includes: (a) preparing a scaffold material solution; (b) air-drying the scaffold material solution, to form a thin scaffold layer; (c) crosslinking the thin scaffold layer; (d) rehydrating the scaffold layer; and (e) applying retinal pigment epithelium cells onto the scaffold layer.

### CROSS REFERENCE

This application is a Continuation in Part patent application of U.S. patent application Ser. No. 18/126,455 filed on Mar. 26, 2023, which is a continuation of U.S. patent application Ser. No. 17/198,449 filed on Mar. 11, 2021. This application also claims the benefit of priority of U.S. Provisional Patent Application No. 63/326,991, filed Apr. 4, 2022 and U.S. Provisional Patent Application No. 63/435,992, filed 29 Dec. 2022, the contents of which are all incorporated herein by reference in their entirety.

### TECHNOLOGICAL FIELD

The present presently disclosed subject matter is in the medical field, in particular treatment of ophthalmic medical conditions/disease.

### BACKGROUND

Corneal blindness is estimated to have effect on the order of 10 million people worldwide. It is further estimated that only about 1% of the people suffering from corneal blindness receive treatment. In part, the low rate of treatment is due to the scarcity of corneal tissue donors. Even in cases in which corneal tissue is available, problems frequently remain, as corneal tissue can easily be damaged during the transplantation procedure, and tends to be less healthy in older donors than in younger ones. Furthermore, the time frame for the donation is quite short, as the tissue should be harvested within several hours of death and transplanted within a few days after harvesting.

The term corneal transplantation is used to describe several different medical procedures, in which different parts of the cornea is being implanted, including: (A) Penetrating Keratoplasty (PK), in which the entire thickness of the cornea is transplanted, (B) Descemet Stripping Endothelial Keratoplasty (DSEK), in which the endothelium membrane, Descemet membrane and part of the posterior corneal stroma are transplanted, and (C) Descemet Membrane Endothelial Keratoplasty (DMEK), in which the endothelium and the Descemet membrane are transplanted, without additional stoma layer.

Among the procedures mentioned above, DSEK is the most common one. In the US, 60% of the total corneal transplantations are DSEK. However, the DMEK procedure has major advantages over it, including lower rejection rate (1% for DMEK compared with 12% of DSEK), higher probability for visual acuity above 0.8 (79% in DMEK compared with 47% in DSEK), and shorter healing period (weeks in DMEK compared with months to year in DSEK).

The main reason that the DMEK procedure is not fully taking over the DSEK is the ease of implantation. In particular, the native DMEK graft tends to break more easily during the procedure, and the endothelial cells in it are sensitive for pressure and shear stresses. Therefore, the DMEK usually requires special implantation techniques and higher experienced surgeons.

In addition, since the DMEK is so fragile and sensitive, doctors prefer stiffer graft obtained from donors older than 50 or 60 years. However, the corneal endothelial cells in these grafts are in a lower density and conditions.

Thus, a reliable source for thin yet stiff corneal implants, with healthy and functioning endothelial cells and repeatable conditions, remains unmet need.

Further, millions of people around the world suffer from retinal degenerative diseases in varying degrees of vision loss including complete blindness. The most common retinal degenerative disease is Age-related Macular Degeneration (AMD). Retinal degeneration affects individuals mostly over the age of 55. Currently, over 200 million people worldwide are affected by AMD, with approximately 10% in an advanced disease stage. Since aging is one of the main risk factors for AMD, these numbers are likely to increase dramatically as life expectancy continues to rise.

AMD has two advanced stages: dry AMD, or geographic atrophy (GA); and wet AMD, or choroidal neovascularization also called NVAMD.

Histologically, the dry form of AMD is characterized by the death of retinal pigment epithelium (RPE) cells in the macular region of the eye, which is the central area of the human field of vision, providing the high-definition vision needed for face recognition, driving, etc. The disease etiology starts with death of the RPE cells that provide photoreceptors (PRs) with vital and functional support. As the disease progresses, there is irreversible photoreceptor cell loss affecting central vision (in the macular region), making patients handicapped. Wet AMD is commonly treated with monthly antiangiogenic injections into the eye, inhibiting blood vessel growth.

In the early stages of dry AMD and for the advanced form of AMD known as geographic atrophy, there is presently no regulatory-approved treatment.

One approach that may be able to provide a treatment for dry AMD is cell therapy. Cell therapies for dry AMD may involve the implantation of retinal pigment epithelium (RPE) cells, and possibly also photoreceptor cells in the advanced stages, when the photoreceptors are lost. There are several RPE cell therapies in various stages of development, either as a suspension or as a monolayer implant, aimed at curing AMD (for a review see: Sharma R, Bose D, Maminishkis A, Bharti K. “Retinal Pigment Epithelium Replacement Therapy for Age-Related Macular Degeneration: Are We There Yet?” Annu Rev Pharmacol Toxicol. 2020 Jan. 6; 60:553-572). Sharma also discloses devices for the delivery of retinal implants.

An additional intervention investigated for the treatment of dry AMD uses medications aimed at the complement immune system, which is hypothesized to lead to the initiation of dry AMD (for a review on this approach see: Retina Today, Therapies on the Horizon for Nonexudative AMD, May/June 2021, https://retinatoday.com/articles/2021-may-june/therapies-on-the-horizon-for-nonexudative-amd).

One of the main approaches of cell therapy for AMD is the injection of RPEs in suspension into the sub-retinal space between the native RPE layer and the PR cell layer. Nevertheless, sub-retinal cell suspension injections run the risk for unwanted distribution of the cells within the eye, which may cause cell adhesion and growth on other parts of the eye such as the intraocular lens or the retinal anterior side. Additionally, there is a risk of loss of viability of the transplanted cells as the microenvironment they are welcomed by in the degenerated eye is hostile.

The most common treatment for wet AMD is intravitreal injection of anti-vascular endothelial growth factors (anti-VEGF). This treatment is a continual procedure, which has to be performed several times per year, and does not cure dystrophy of the RPEs. This treatment also suffers from risks of endophthalmitis, intraocular hypertension, chronic eye pain, and more (see: Review of Ophthalmology, Safety of Intravitreal Anti-VEGF Agents, 11 Nov. 2014, https://www.reviewofophthalmology.com/article/safety-of-intravitreal-antivegf-agents).

There are additional eye diseases in which RPE cell issues are the leading cause of vision loss, due to genetic hereditary malfunctional genes in the RPE cells such as Best and Stargart disease.

Additionally, in a degenerated retina, the PR cells are either dead or not functioning, leading to blindness or impaired vision. Some attempts to make an electronic retinal prosthesis were made in the recent years, showing the ability to provide a very limited degree of vision, although with the risk of additional retinal damage.

### GENERAL DESCRIPTION

The present presently disclosed subject matter relates to retinal grafts or implants; and methods of preparing such grafts/implants.

According to one aspect of the presently disclosed subject matter there is provided a retinal graft or implant. The graft/implant includes a scaffold made of a protein material, such as collagen, or a synthetic chemical; and a layer of matured retinal pigment epithelium (RPE) cells positioned on top of the scaffold or integrated within the scaffold.

Herein the specification and claims, the terms graft, implant and patch, RPE patch, and retinal patch and their derivatives, may be used interchangeably. Likewise, the terms film, scaffold, collagen layer, support, and substrate may be used herein interchangeably in the specification and claims.

In some examples, the graft/implant further includes photoreceptor (PR) cells attached to the RPE cells or integrated between them.

Unlike cell suspension injections, transplanting the RPE cells in an organized and matured retinal graft delivers fully differentiated, polarized, confluent (i.e. significantly covered/high density coverage of cells) and functional cells, with a preexisting microenvironment allowing them to integrate in the patient's eye with high viability and low risk of unwanted distribution.

According to another aspect of the presently disclosed subject matter there is provided a method of preparing a retinal graft/implant. The method includes preparing a scaffold, made using collagen, or other suitable protein, or a synthetic (i.e. non-protein chemical) material; applying a layer of matured retinal pigment epithelium (RPE) cells on or within the scaffold; and potentially also photoreceptors cells. Unlike cell suspension injections, transplanting the RPE cells in an organized and matured retinal graft delivers fully differentiated, polarized, confluent and functional cells, with a preexisting microenvironment allowing them to integrate in the patient's eye with high viability and low risk of unwanted distribution.

It is further an object of the current invention to disclose the preparation of PVEK, namely a DMEK-like implant, which may be implanted using the detachable and/or dissolvable carrier device mentioned in the previous file.

The terms “Precise vision endothelial keratoplasty” and PVEK for short, will be interchangeably herein after refer to a set of solutions found in a few cases being equal to, if not better than the DSEK implant, as some the inventors disclosed in U.S. Pat. Appl. No. 62/487,018 “Bioengineered corneal graft and methods of preparation thereof” incorporated herein as a reference. DMEK is configured to be implanted without a carrier and the carrier can be used with native DMEK grafts.

The PVEK-implant of the present invention is very thin, but is still stiff enough to handle with standard ophthalmic tools. Also, native DMEK graft tend to fold due to the contraction of the cells on the Descemet membrane. This makes the implantation much more difficult as the surgeon has to flatten the graft in the eye. Here however, the membrane does not tend to fold and the graft opens more easily after insertion.

The PVEK-grafting by the present technology comprises, inter alia, step(s) of drying out a solution of collagen and/or gelatin on a surface, and crosslinking it with EDC/NHS solution, that bond the collagen and/or gelatin polymers and make it a hydrogel thin film. Since the crosslinking process occurs while the material is not fully wetted, the resulting hydrogel is very thin and with high polymer concentration. These layers can be implanted in a similar way to DMEK, but have an advantage that they do not tend to fold after implantation like DMEK.

The present invention hence discloses, inter alia, an artificial endothelial keratoplasty graft consisting a support layer made of rehydrated crosslinked hydrogel and corneal endothelial cells on top or within said support layer.

The present invention also discloses an artificial endothelial keratoplasty graft as defined above, wherein the rehydrated crosslinked hydrogel is based on crosslinked collagen or collagen methacrylate.

The present invention also discloses an artificial endothelial keratoplasty graft as defined in any of the above, wherein the source of said collagen or collagen methacrylate is human recombinant collagen.

The present invention also discloses an artificial endothelial keratoplasty graft as defined in any of the above, wherein the rehydrated crosslinked hydrogel is based on crosslinked gelatin or gelatin methacrylate.

The present invention also discloses an artificial endothelial keratoplasty graft as defined in any of the above, wherein the total thickness of the graft is between 5 and 50 microns.

The present invention also discloses an artificial endothelial keratoplasty graft as defined in any of the above, wherein the total thickness of the graft is below 25 microns, therefore mimicking native DMEK graft.

The present invention also discloses an artificial endothelial keratoplasty graft as defined in any of the above, wherein the mechanical properties of said support layer allows positioning and flattening of the graft inside the anterior chamber during implantation.

The present invention also discloses an artificial endothelial keratoplasty graft as defined in any of the above, wherein the cells remodeled the said support layers to form corneal endothelium extra-cellular matrix, by a long maturation period or by additional nutritions to the cells.

The present invention further discloses a method of manufacturing an artificial endothelial keratoplasty graft, wherein the method consisting of a step of drying support layer material followed by a crosslinking step.

The present invention also discloses the method as defined above, wherein the support layer material is collagen or collagen methacrylate.

The present invention also discloses the method as defined in any of the above, wherein the source of said collagen or collagen methacrylate is human recombinant collagen.

The present invention also discloses the method as defined in any of the above, wherein the support layer material is gelatin or gelatin methacrylate.

The present invention also discloses the method as defined in any of the above, wherein the crosslinking step involves in introducing EDC and NHS molecules to the material.

The present invention also discloses the method as defined in any of the above, wherein the crosslinking step involves in introducing LAP or 1-[4-(2-Hydroxyethoxy)-phenyl]-2-hydroxy-2-methyl-1-propane-1-one (a commercially available as Irgacure 2959 trademark by Ciba) molecules to the material and applying light on it.

The present invention also discloses the method as defined in any of the above, wherein the drying step is performed in controlled environmental conditions.

The present invention also discloses the method as defined in any of the above, wherein the final thickness of said graft is below 25 microns.

The present invention also discloses the method as defined in any of the above, wherein the mechanical properties of said graft allow pulling and pushing it during implantation, therefore allowing positioning and flattening inside the anterior chamber of the eye.

The present invention also discloses the method as defined in any of the above, wherein the water content in said support material is between 30 and 90 percent.

### BRIEF DESCRIPTION OF THE DRAWINGS

The presently disclosed subject matter may be more clearly understood upon reading of the following detailed description of non-limiting exemplary examples thereof, with reference to the following drawings, in which:

FIG. 1 schematically illustrating a DMEK-like graft according to one embodiment of the invention;

FIG. 2 schematically illustrating a DMEK-like graft on a designated carrier according to another embodiment of the invention; and

FIG. 3 schematically illustrating a corneal graft orientation in the eye, according to yet another embodiment of the invention.

FIG. 4 is a schematic drawing of the human retina anatomy.

FIG. 5 is a schematic drawing of a retinal graft, in accordance with the disclosed subject matter.

FIG. 6 is a schematic depicting an exemplary method of preparing a scaffold layer of the retinal graft, in accordance with the disclosed subject matter.

FIG. 7 is a schematic depiction of RPE cells positioned on top and within the scaffold.

FIG. 8 is a photo of two retinal implants in a pig's eye.

FIG. 9 is a histology of a retinal implant, 2 months post implantation in a pig's eye in vivo.

FIG. 10 is a fluorescent imaging photo of an in vitro retinal implant with polygonal monolayer of RPE cells.

The following detailed description of examples of the presently disclosed subject matter refers to the accompanying drawings referred to above. Dimensions of components and features shown in the figures are chosen for convenience or clarity of presentation and are not necessarily shown to scale. Wherever possible, the same reference numbers will be used throughout the drawings and the following description to refer to the same and like parts.

### DETAILED DESCRIPTION OF THE INVENTION

Illustrative examples of a retinal graft and method of preparing it according to the presently disclosed subject matter are described below. In the interest of simplicity, not all features/components of an actual implementation are necessarily described.

The artificial Endothelial Keratoplasty Implant and methods of preparation thereof disclosed herein are designed to meet this need, by allowing the production of DMEK-like grafts with healthy and functioning cells and good mechanical properties.

The implant is made of Descemet membrane-like hydrogel support layer, and a layer of corneal endothelial cells (CEC) layer on top. The endothelial layer is made of CEC cells obtained from donors' corneas and proliferated, or by stem-cells differentiation, by methods known in the art.

The support layer is made of a thin collagen or gelatin layer, which was designed to have mechanical properties, transparency and dimensions to improve patients' vision and health condition.

To produce the endothelial keratoplasty, a layer of low concentration collagen solution is spread on a surface and dehydrated. After drying out, a crosslinker solution is introduced to the dried material which form a crosslinked hydrogel in thicknesses between 2 to 50 micrometers. The excess and un-crosslinked reagents are washed away using PBS and corneal endothelial cells are seeded on top of the hydrogel, forming a two-layer construct, similarly to DMEK cadaver grafts.

In some cases, the corneal graft is implanted onto the posterior cornea using a designated DMEK tools or carrier. In preferred embodiments, prior to the implantation a carrier layer is attached to the corneal graft using biocompatible adhesive material, forming a 4-layers implant: An Endothelial keratoplasty graft made of a collagen/gelatin layer and a cells layer, an adhesive layer, and a carrier. After implantation, the adhesive and the carrier layer are possibly detached from the graft and pulled out or dissolved in the eye.

In other cases, the corneal graft is implanted similarly to cadaver-sourced DMEK grafts, by methods known in the art.

The present invention hence discloses, inter alia, an artificial endothelial keratoplasty graft consisting a support layer made of rehydrated crosslinked hydrogel and corneal endothelial cells on top or within said support layer.

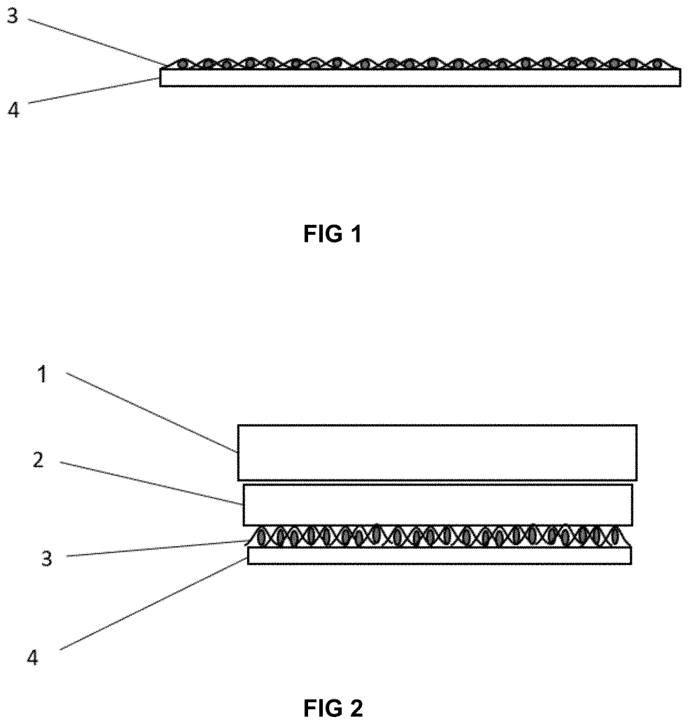

Reference is made to FIG. 1, schematically illustrating a DMEK-like graft according to one embodiment of the invention. Upper layer is corneal endothelial cells (3) and below is a support layer (4).

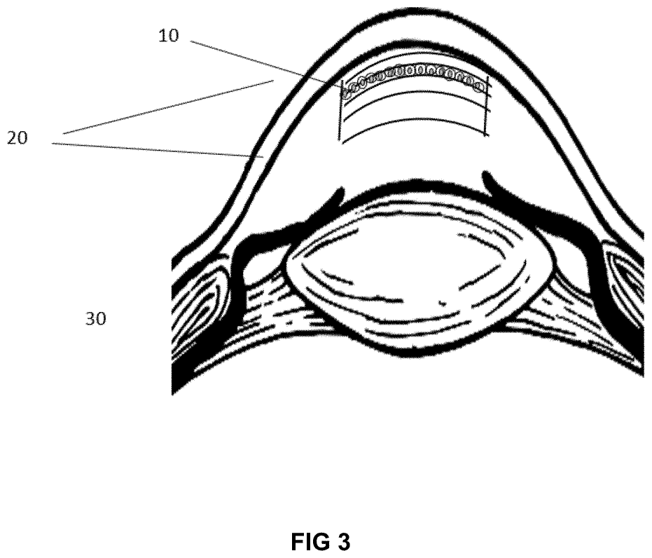

Reference is now made to FIG. 2, schematically illustrating a DMEK-like graft on a designated carrier according to another embodiment of the invention. upper layer in this illustration is a carrier (1); and below are adhesive (2), Corneal endothelial cells (3) and support layer (4).

Reference is now made to FIG. 3, schematically illustrating a corneal graft orientation in the eye, according to yet another embodiment of the invention. Upper illustration is a corneal graft with adhesive and carrier layers attached to the posterior cornea (not in scale) (10). Cornea (20) and anterior chamber (30) are also illustrated. Figure is adapted from a currently available public draw https://www.iconspng.com/image/92595/eye-3.

It is according to a few embodiments of the invention wherein the graft comprises two main layers, namely (i) a support (Descemet membrane-like) layer, made of materials selected from a group consisting of collagen, gelatin, collagen methacrylate, gelatin methacrylate and a combination thereof; and (ii), a corneal endothelial cells layer, seeded on top of the support layer.

It is according to a few embodiments of the invention wherein the geometry of the support layer is ranging between about 2 and about 50 microns thick; diameter ranging between about 7.5 to about 9.5 mm.

It is further according to a few other embodiments of the invention wherein the geometry of the cells layer is ranging between about 2 and about 20 microns in thickness, cells density of about 1,500 to about 4,000 cells/mm\hat{ }2; diameter of about 7.0 to about 9.5 mm.

It is according to a few embodiments of the invention wherein the materials of the support layer are selected from a group consisting of Collagen, Collagen methacrylate, Gelatin, Gelatin from about 1 to about 50% w/v., whereas native stroma has 13% collagen.

It is according to a few embodiments of the invention wherein additional materials of the support layer are selected from a group consisting of photo-initiators and crosslinkers: LAP, Irgacure 2959, APS-TEMED, 1-ethyl-3-(3-dimethylaminopropyl) carbodiimide (EDC), N-hydroxysuccinimide (NHS), and a combination thereof.

It is according to a few embodiments of the invention wherein additional materials of the support layer are selected from a group consisting of biocompatible dyes for easy handling such as trypan blue.

It is according to a few embodiments of the invention wherein the materials of the cells layer are selected from a group consisting (i) human corneal endothelial cells from human donors, with or without expansion of the cells by a proliferation step; (ii) human corneal endothelial cells derived from stem cells such as induced pluripotent stem cells (iPSC) or human embryonic stem cells (hESC); (iii) cells own ECM, produced by the cells during the maturation process and any combination thereof.

It is according to a few embodiments of the invention wherein the preparation methods comprise drying step(s). Such a drying steps comprises steps of spreading or molding about 0.1 to about 15% (w/v) materials selected from collagen, collagen methacrylate, gelatin, and gelatin methacrylate on a surface. The spread/molded layer thickness may vary between about 0.1 and about 10 mm. Then, the materials are let to dry at specific range of temperatures, between about 4 and 60 degrees Celsius, and relative humidity levels. Total drying period time is ranging from 1 h to about 10 days. The drying process may be consisted of several different environmental condition steps.

It is according to a few embodiments of the invention wherein the preparation methods comprise crosslinking step(s). After drying, crosslinking the dried sheet by washing it with one or more of the following: EDC, NHS, LAP, Irgacure 2959 or other crosslinker solution, or a mixture of the above. Using photo-initiating light source is possible to induce the process. Crosslinking time between about 1 minute and about 48 hours. Afterwards, washing the crosslinked gel to remove residues of crosslinker and un-crosslinked polymer and immersing it in water/PBS/media. Then, possibly repeating the drying and crosslinking steps multiple times.

The present invention discloses a method of manufacturing an artificial endothelial keratoplasty graft, wherein the method consisting of a step of drying support layer material followed by a crosslinking step.

It is according to a few embodiments of the invention wherein the preparation methods comprise seeding step(s). Seeding corneal endothelial cells on top of the gel. Maturing for about 1 to about 28 days in an incubator.

### Example 1

Spreading 1604 of 0.5% (w/v) collagen on an area of 10 mm×10 mm, on a flat surface (e.g. Petri dish), to form a layer of about 2 mm thick. Letting the material dry at 4 degrees Celsius, 40% RH, for 48 hours. Adding 2 ml PBS solution with (1 mg/ml) EDC and (0.25 mg/ml) NHS, and let the material crosslink for 2 h at 25 degrees Celsius. Rinsing the gel 3 times in PBS, with final wash of 12 hours in PBS. Pipetting out the PBS and seeding CECs on the hydrogel surface.

### Example 2

Spreading 1004 of 5% (w/v) gelatin methacrylate on an area of 10 mm×10 mm, on a flat hydrophobic plastic surface (e.g. Petri dish), to form a layer of about 1.2 mm thick.

Letting the material dry in a 25 degrees Celsius vacuum desiccator for 12 hours. Adding 2 ml PBS solution with (0.5% w/v) LAP, and applying UV light at 1 mW/cm\hat{ }2 to crosslink the material for 30 minutes. Rinsing the gel 3 times in PBS, with final wash of 12 hours in PBS. Pipetting out the PBS and seeding CECs on the hydrogel surface.

It is according to a few embodiments of the invention wherein grafts and implanted via a few optional procedures, see examples 3-4.

### Example 3

The artificial EK graft is implanted using standard DMEK or DSEK techniques and tools, such as Coronet Endoglide (commercially available by Network Medical Products) or Geuder cannula. This includes loading the graft onto the tool (before or after shipment), inserting the tool through a peripheral corneal incision, injecting/pulling/pushing the graft into the patient's anterior chamber, and attaching the graft to the posterior cornea with an air bubble.

### Example 4

To make the implantation procedure easier and safer, a designated carrier device is used to insert the graft into the patient's eye (see FIG. 2). The carrier is made of detachable/dissolvable thermo-responsive materials, with a diameter which is similar to the graft or slightly larger. The EK graft is attached to the carrier device, folded and inserted into the eye. After insertion, an air bubble is injected to attach the graft to the posterior cornea. Then, the cornea is warmed to 34-38 degrees Celsius to detach or dissolve the carrier.

### Example 5—Crosslinking Methods

### Drying the Collagen

a. According to the number of films, Put collagen 0.05% to 5% in a tube, unscrew cap lightly and place in desiccator under vacuum for outgassing.
b. Pipette 100-900 μl collagen in dedicated molds.
c. Dry the collagen in the molds for 1-7 days in laminar hood thereby producing films.

### Cross-Linking the Films:

a. Prepare cross linking solution:
   i. Take out 1-ethyl-3-(3-dimethylaminopropyl) carbodiimide, EDC, and N-hydroxysuccinimide, NHS.
   ii. Prepare solution of EDC and NHS in Ethanol 100%.
b. Filter the solution
c. Put up to 2 mL of cross-linking solution in each of the molds.
d. Incubate up to 4 h at RT in laminar hood.
e. Remove cross linking solution.
f. Wash with PBS.
g. Store until use.

It is another object of the present invention to produce the retinal graft a scaffold can be produced using either a biological or synthetic material.

Biological materials (i.e. proteins usually sourced from animals or cadavers) may include one or more of the following: collagen types I, III, and W, gelatin, alginate, laminin, fibronectin, Matrigel (a solubilized basement membrane matrix secreted by Engelbreth-Holm-Swarm (EHS) mouse sarcoma cells), silk fibroin, hyaluronic acid and vitronectin. Possible sources for the biological collagen material include human cadavers; animals; and recombinant sources such as fungi, bacteria or plants.

Synthetic materials may include: poly(lactic-co-glycolide acid) (PLGA), poly(l-lactic acid) (PLLA), PLLA-PLGA copolymer systems, poly(glycerol-sebacate) (PGS), polydimethylsiloxane (PDMS), polydimethylsiloxane (PDMS), poly(methyl methacrylate) (PMMA), poly(ethylene glycol) diacrylate (PEGDA), parylene-C and polycaprolactone (PCL), or combinations thereof.

The above-mentioned biological and synthetic materials can be processed by crosslinking, drying, molding, casting, printing, vitrification, polymerization, compression, freeze-drying, laser ablation, laser patterning, melting, evaporation, condensation or a combination of the above.

Addition active substances may be added to the scaffold, including slow-release medication, dyes, anti-VEGF drugs, steroids, and antibiotics.

As noted, the RPE cell layer may be positioned on top of the retinal implant scaffold, or integrated within the scaffold. The RPE cells can be arranged in a monolayer, with physical connection and biological interactions between the RPE cells, or they can be positioned separately within the scaffold, the cells being surrounded by or embedded in the scaffold.

The RPE and PR cells can be sourced from cadavers, or produced from stem cells. The most common source for retinal cells is differentiated human embryonic stem cells (hESCs); however, other cells types can be used, including induced pluripotent stem cells (iPS) from an autologous or allogeneic source; mesenchymal stem cells; and somatic stem cells.

In some preferred examples, the scaffold is made by air drying a collagen solution, followed by crosslinking and rehydrating steps, which is somewhat similar to the process disclosed in patent application IL 269671, which relates to corneal endothelial cells, and not retinal pigment epithelium (RPE) cells; however, there are significant/non-trivial differences in the methodologies and use. Such as collagen source/type; thickness; concentration; crosslinker; crosslinking conditions; and thickness of the scaffold.

The step of air drying the collagen is done by adding a predefined volume of aqueous collagen solution to a mold, setting the desired environmental conditions, such as humidity, temperature, gas composition, gas flow rate and direction, and light radiation, and letting the solution dry for a predefined period of time. The dried collagen is then crosslinked by adding a liquid crosslinking solution to the mold, or by immersing the dried collagen in a crosslinker liquid solution. In some examples, there is an alternative or additional step of photo crosslinking.

The crosslinking solutions may be based on water; saline; an aqueous buffer solution; an alcohol solution, an alcohol-water solution; or a mixture thereof. Following the crosslinking step, the crosslinked collagen film is rehydrated and washed with an aqueous solution such as phosphate buffer saline (PBS) (e.g. a saline solution with proteins and glucose added); or RPE/PR cell medium.

This method produces a membrane of collagen, which has several advantages over other retinal implant scaffolds, including at least one of the following: (a) good support for the RPE cells, with good cell attachment; (b) chemical stability for long periods in aqueous solutions and in an RPE/PR cell medium at human body temperature; (c) a thin membrane that mimics the Bruch's membrane, usually 2-20 μm thick (and preferably 5-10 μm thick); (d) flexibility to allow implantation into the sub-retina through a small retinotomy; (e) resistance to tearing, to protect the cells and ease implantation; (f) high permeability to proteins; glucose; and oxygen, to provide good nutrient supply from the vascular system to the cells; and (g) a basis on natural proteins to either recognize the scaffold as a natural self-tissue or optionally facilitate remodeling by the implanted cells, or by the patient's post cell implantation. The idea is that in many cases one wants the scaffold to degrade or become natural tissue after it is implanted into the eye. This is done by nearby cells, which consume the proteins (collagen), and possibly produce other proteins. This process can be done by the added cells to the scaffold and implanted, or by the patient own cells.

To produce the crosslinked, rehydrated collagen scaffolds, a controlled environment during the drying process is used, which may include a 10%-99% relative humidity, air flow, 4-40 degree Celsius temperature range; and gas composition. Additionally, an appropriately designed drying mold is required to produce a suitable and uniform scaffold, the mold having wetting properties that may be hydrophilic surface or having a hydrophobic surface; e.g. made of glass; plastic, such as PTFE; metal; ceramic, or combination thereof, and made of one or more parts. The mold may comprise coatings thereon. The mold may have a wide range of geometries (flat, concave, convex, etc.) that can be beneficial for interfacing. The mold may also be designed (e.g. with a nozzle, inlet tube or the like) to allow and/or support and/or direct air or gas flow to aid in the drying process.

In some preferred examples, the air-dried collagen scaffold is crosslinked by an NHS-EDC (NHS: N-hydroxysuccinimide; EDC: 1-ethyl-3-(3-dimethylaminopropyl)carbodiimide hydrochloride) solution, at a specific concentration as known per se, within a temperature range of 0-40 degrees Celsius and for a period of time of 1 minute to 24 hours.

In other examples, the air-dried collagen scaffold is crosslinked using enzymes such as transglutaminase; or by applying UV light; or by using elevated temperature (35-90 degrees Celsius); or by chemical agents as glutaraldehyde.

If the air-dried collagen scaffold adheres to the mold surfaces, one or more additional steps may be required to peel it off or otherwise remove the scaffold, before or after the crosslinking step. This removal may include washing with a water or alcohol solution; a secondary drying step after crosslinking, using a low surface-tension solution; cutting the scaffold; or using special tooling such as tweezers, spatulas, vacuum sockets, an air blower, or the like.

An exemplary method of preparing the retinal implant of the disclosed subject matter is as follows:
(a) Aqueous protein solution is placed on/in a mold.
(b) The solution is left to air-dry at the suitable environmental conditions and time period.
(c) Crosslinker solution is added to the mold to produce a cross-linked film (scaffold).
(d) The film is extracted from the mold.
(e) The film is washed with PBS (saline) or cell-media or water, of any combination thereof
(optional) The film is cut to the desired or a predetermined size.
(optional) The film is air-dried again.

RPE cells are seeded (poured/printed) on the scaffold/film.

The RPE cells are allowed to settle and attach to the scaffold for approximately 0.5 to 6 hours producing an RPE cell implant.

The RPE cell implant is placed in a culture dish and RPE cell media is added to the dish, which is then covered.

The implant is incubated for 1-30 days for maturation.
(Optional) The graft is cooled to cryogenic temperatures in the middle of the maturation period, and then thawed for further maturation. For implanting, the resultant implant is loaded into an implantation tool

### Example

1 ml of 3 mg/ml collagen solution was added to a mold (e.g., glass mold), having a round flat bottom and diameter of 5 mm. The (uncovered) mold with the collagen solution was then placed in a 37 C, 90% RH chamber, with no air flow, for 10 days. 2 ml of EDC aqueous solution was added to the mold, and kept for 24 hours, at 37 C. The EDC solution was removed. A PBS solution was then added to the mold to wash off any cross-linker residue. The resultant film was detached from the mold using a PBS solution. The separated film was washed 3 times with PBS, and immersed in a cell media for 10 minutes. The film was then positioned on a glass cover slip, and placed in a well plate. The well plate was inserted into a laser-induced-forward transfer (LIFT) bioprinting system, and RPE cells were printed on the film at a density of about 2000 cells per mm\hat{ }2. The printed scaffold with RPE cells were kept in a humid chamber for 4 hours to allow the cells to form attachments to the scaffold (humidity is critical to avoid drying of the graft). After 4 hours, additional cell media was added to the well plate, covering the graft. Then the graft was placed in an incubator for 21 days. At day 22, the graft was cryo-frozen by replacing the media with cryopreservation substances, and placing it in a minus 80 C freezer (allowing extended preservation, e.g. for up to several months).

When needed, the graft can be slowly thawed, followed by replacing the cell media, and performing a secondary maturation incubation period, of 1-5 days.

The crosslinking step involves in introducing lithium phenyl-2,4,6-trimethylbenzoylphosphinate (LAP) or 1-[4-(2-Hydroxyethoxy)-phenyl]-2-hydroxy-2-methyl-1-propane-1-one (commercially available as Irgacure 2959, a trademark by Ciba Vision Corp.) molecules to the material and applying light on it.

In some preferred examples, the retinal pigment epithelium cells are seeded (applying cells and letting them attach—chemically/biologically) on the substrate (on the scaffold surface), and allowed to sit for 1-4 hours to let the cells adhere to the scaffold.

In some examples, the seeding step is done by pipetting. In other examples, the seeding is performed by printing, in particular by inkjet printing, micro-pipetting or laser-induced-forward-transfer (LIFT) printing. Printing the cells can provide better cell density uniformity on the scaffold surface, better cell attachment, and less clumping or aggregation.

In some preferred examples, following the seeding or printing step, the graft is immersed or covered in a cell medium and incubated for maturation. The composition of the cell medium is designed specifically for the type of cells and to promote maturation of the implant. In some cases, the medium may include antibiotics, steroids, dyes, buffers, serum or a combination thereof.

The maturation step can be 3-60 days long. In some preferred examples, the maturation period is 21-28 days long.

Before or after cell seeding and maturation, the retinal implant may be cut to a size that can fit on an insertion tool, size typically being 1-6 mm in diameter. The cutting step can be performed by punching, knife-cutting, a tissue slicer (a guillotine-like device), laser ablation or scissors.

Additionally, a mark to differentiate the orientation of the graft/implant can be added/applied thereto, to ensure that the cells are positioned toward the PR layer after implantation, and not toward the native RPEs layer. This mark can be performed using staining, pattering (patterned staining, e.g., coloring the entire structure, or “writing” a symbol on it with a stain, commonly performed using an S-shaped medical marking tool), or cutting the implant, before or after cell seeding and maturation.

In some examples, the grafts are frozen before, after or in the middle of the maturation step. The freezing step can be performed by methods known in the art such as cryopreservation; slow freezing; vitrification; liquid nitrogen freezing; using a freezing medium containing DMSO, or a combination of the above. Frozen grafts can be preserved for long periods of time, even years, allowing QC tests to be performed on the grafts and on cell batches before implantation into a patient's eyes. For clarification, the term “cell batches” refers to cells produced by a batch process—produced in bio-reactors, and harvested as a batch every few days. To ensure there are no toxins/pathogens or the like, it is preferable to freeze most of the cells from each batch, to test the unfrozen cells, and then unfreeze the frozen cells.

One of the main problems to overcome by the retinal implant method versus cell suspension is the delivery of the graft, which requires a much larger port than when injecting cells in a suspension into the subretinal space. Therefore, in some preferred examples, the retinal implant uses a suitable delivery device, which will support the scaffold and the cells of the retinal patch during the implantation procedure.

The retinal graft may be implanted into a patient's eye by one or more of the following:
1. 3 sclerotomies (opening ports in the sclera) and placing trocars
2. Pars plana core vitrectomy, performed using a constellation vitrectomy system
3. intravitreal (triamcinolone acetonide) injection
4. posterior vitreous detachment (PVD) induction (if needed)
5. producing a sub-retinal pocket, by starting to elevate a bleb outside the macula
6. creation of a temporal retinotomy after a diathermia
7. inserting of an implantation device into the retinotomy
8. retinal implant delivery into the sub retina
9. flattening the retina using Perfluorocarbon (PFC) heavy liquid
10. intraocular laser retinopexy around the retinotomy site
11. removing of the PCF from the eye
12. fluid air exchange
13. injecting silicone oil into the eye
14. removal of trocars and suturing of sclerotomies

Thus, this disclosure provides a safe and reliable retinal graft and method of preparing same, where the RPE cells can be delivered as an organized and highly viable and functional implant with, and potentially integrate and support any surviving PR cells to provide a treatment for dry AMD.

### Example 1—Crosslinking

Preparation of the Films:
1. Add 50-10000 collagen (0.3% in HCl 10 mM) to a mold, gently to avoid spilling.
2. Leave to dry in hood for 24-96 h.

Cross-Linking the Films:
1. Prepare cross linking solution:
   a. Take out 1-ethyl-3-(3-dimethylaminopropyl) carbodiimide, EDC, and N-hydroxysuccinimide, NHS,
   b. Prepare initial solutions of 0.1-100 mg/mL EDC in Ethanol 100% and 0.1-100 mg/mL NHS in Ethanol 100%.
   c. Mix 1:1 before use to reach final solution of 0.05-50 mg/mL EDC and 0.05-50 mg/mL NHS.
   d. Put 1 mL of cross-linking solution in each mold.

Finalizing the Films and Punching the Same:
1. Punch films to 4-20 mm diameter circles, using a designated punch.
2. Add ˜2 mL of cross-linking solution and cover.
3. Incubate for 0.5-4 h at RT.
4. Remove solution and add PBS in excess (>2 mL). Repeat washing several times (at least 5 min each wash).
5. Keep in 4° C. until use.

Alternatively, the step of punching the films is performed after the step of adding ˜2 mL of cross-linking solution and cover.

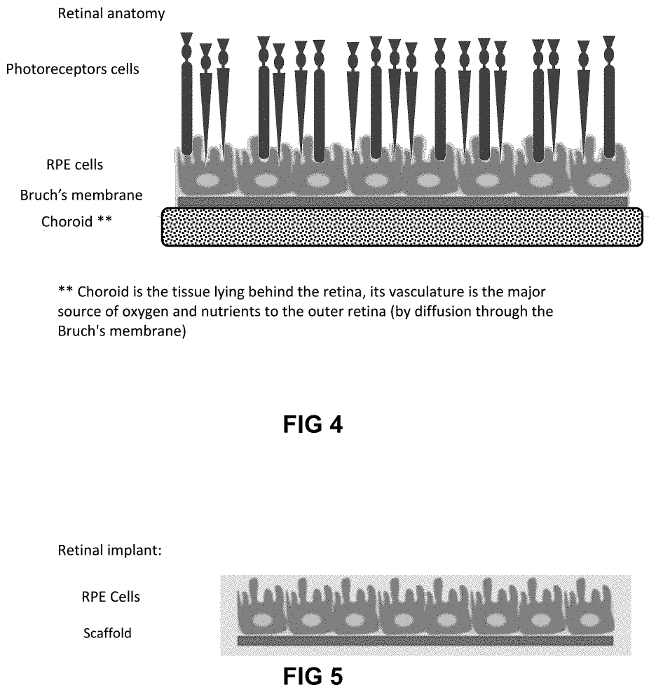

Reference is now made to FIG. 4 illustrating the human retina anatomy. Viewable in the Figure are photoreceptors cells, RPE cells, Bruch's membrane and the Choroid. It is noted that Choroid is the tissue lying behind the retina, its vasculature is the and major source of oxygen and nutrients provide blood flow to the outer retina (by diffusion through the Bruch's membrane).

Reference is now made to FIG. 5 illustrating a retinal graft, in accordance with the disclosed subject matter. As seen in the figure, the graft comprises the RPE cells on the Scaffold.

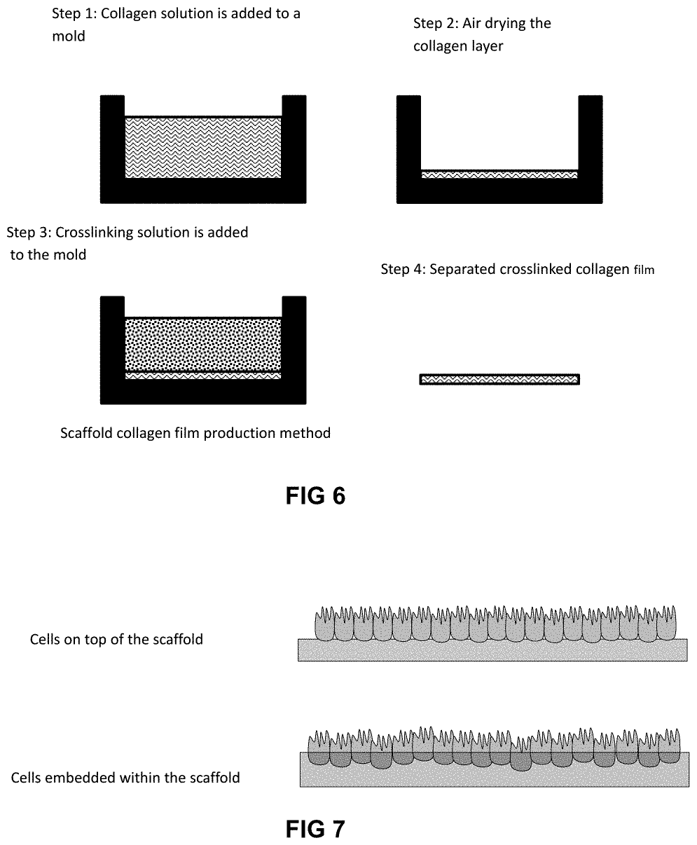

Reference is now made to FIG. 6 illustrating an exemplary method of preparing a scaffold layer of the retinal graft, in accordance with the disclosed subject matter. As disclosed above, in Step 1: Collagen solution is added to a mold; in Step 2: Air drying the collagen layer; in Step 3: Crosslinking solution is added to the mold; in Step 4: Separated crosslinked.

Reference is now made to FIG. 7 illustrating the RPE cells positioned on top and within the scaffold.

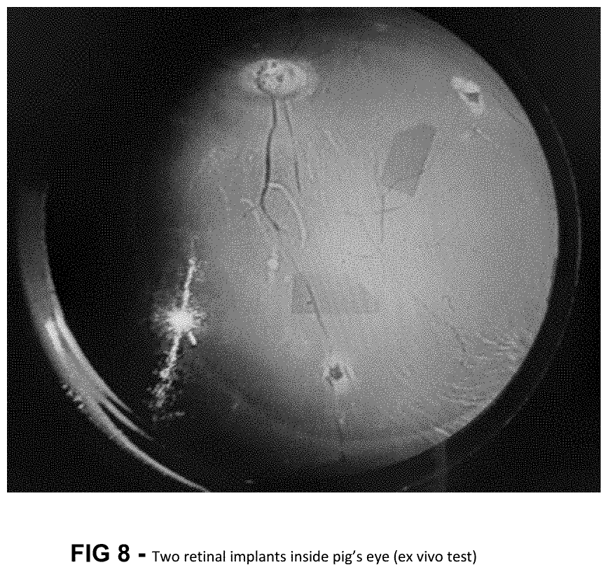

Reference is now made to FIG. 8 illustrating is a photo of two retinal implants in a pig's eye. As seen in the figure, two retinal implants inside pig's eye (ex vivo test).

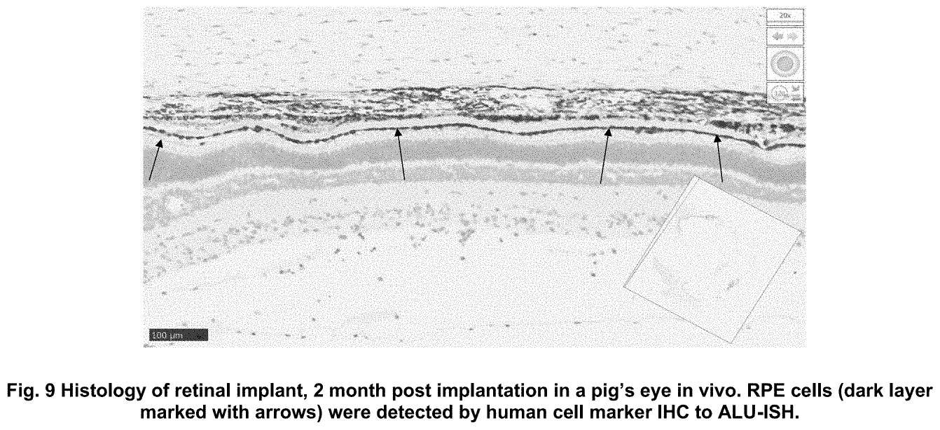

Reference is now made to FIG. 9 illustrating a histology of a retinal implant, 2 months post implantation in a pig's eye in vivo. RPE cells (dark layer marked with arrows) were detected by human cell marker IHC to ALU-ISH.

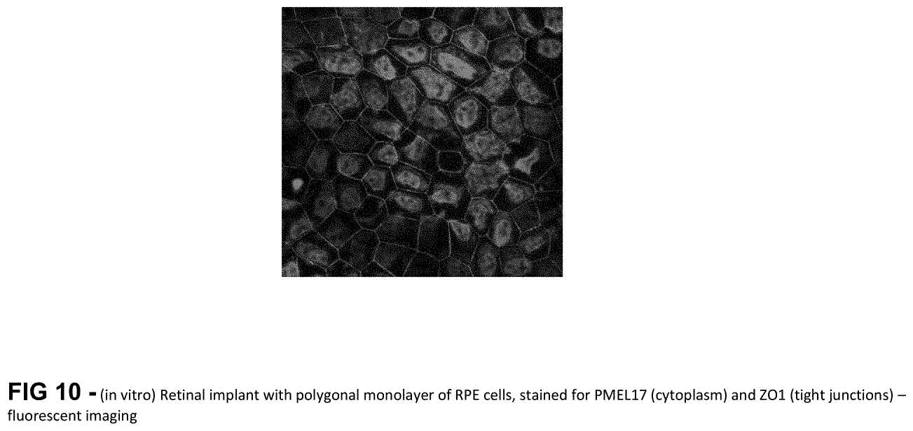

Reference is now made to FIG. 10 which is a fluorescent imaging photo of an in vitro retinal implant with polygonal monolayer of RPE cells. The figure illustrates an in vitro retinal implant with polygonal monolayer of RPE cells, stained for PMEL17 (cytoplasm) and ZO1 (tight junctions)—fluorescent imaging.

All references cited throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, and method steps set forth in the present description. As will be obvious to one of skill in the art, methods and devices useful for the present methods can include a large number of optional composition and processing elements and steps.

When a group of substituents is disclosed herein, it is understood that all individual members of that group and all subgroups, including any isomers, enantiomers, and diastereomers of the group members, are disclosed separately. When a Markush group or other grouping is used herein, all individual members of the group and all combinations and sub-combinations possible of the group are intended to be individually included in the disclosure. When a compound is described herein such that a particular isomer, enantiomer or diastereomer of the compound is not specified, for example, in a formula or in a chemical name, that description is intended to include each isomer and enantiomer of the compound described individually or in any combination. Additionally, unless otherwise specified, all isotopic variants of compounds disclosed herein are intended to be encompassed by the disclosure. For example, it will be understood that any one or more hydrogens in a molecule disclosed can be replaced with deuterium or tritium. Isotopic variants of a molecule are generally useful as standards in assays for the molecule and in chemical and biological research related to the molecule or its use. Methods for making such isotopic variants are known in the art. Specific names of compounds are intended to be exemplary, as it is known that one of ordinary skill in the art can name the same compounds differently.

It must be noted that as used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art, and so forth. As well, the terms “a” (or “an”), “one or more” and “at least one” can be used interchangeably herein. It is also to be noted that the terms “comprising”, “including”, and “having” can be used interchangeably. The expression “of any of claims XX-YY” (wherein XX and YY refer to claim numbers) is intended to provide a multiple dependent claim in the alternative form, and in some embodiments is interchangeable with the expression “as in any one of claims XX-YY”.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are described. Nothing herein is to be construed as an admission that the invention is not entitled to antedate such disclosure by virtue of prior invention.

Whenever a range is given in the specification, for example, a range of integers, a temperature range, a time range, a composition range, or concentration range, all intermediate ranges and subranges, as well as all individual values included in the ranges given are intended to be included in the disclosure. As used herein, ranges specifically include the values provided as endpoint values of the range. As used herein, ranges specifically include all the integer values of the range. For example, a range of 1 to 100 specifically includes the end point values of 1 and 100. It will be understood that any subranges or individual values in a range or subrange that are included in the description herein can be excluded from the claims herein. The term “about” refers to any value being lower or greater than 20% of the defined measure.

As used herein, “comprising” is synonymous and can be used interchangeably with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” can be replaced with either of the other two terms. The invention illustratively described herein suitably can be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the invention has been specifically disclosed by preferred embodiments and optional features, modification and variation of the concepts herein disclosed can be resorted to by those skilled in the art, and that such modifications and variations are considered to be within the scope of this invention as defined by the appended claims.

1. A retinal graft comprising:
a scaffold layer made of a rehydrated crosslinked scaffold layer; and
at least one matured layer of retinal pigment epithelium, RPE, cells on top or within said scaffold layer; wherein said scaffold layer was initially dried and only then crosslinked before said at least one matured layer of RPE cells are position on top or within said scaffold layer.

2. The graft of claim 1, wherein the rehydrated crosslinked film comprises at least one selected from a group consisting of collagen type I, collagen type III, collagen type IV, gelatin, alginate, laminin, fibronectin, Matrigel (a solubilized basement membrane matrix secreted by Engelbreth-Holm-Swarm (EHS) mouse sarcoma cells), silk fibroin, hyaluronic acid, vitronectin and any combination thereof.

3. The graft of claim 1, wherein the rehydrated crosslinked film comprises crosslinked collagen or collagen methacrylate.

4. The graft of claim 1, wherein the scaffold comprises human recombinant collagen or collagen methacrylate.

5. The graft of claim 1, wherein the rehydrated crosslinked film comprises at least one selected from a group consisting of poly(lactic-co-glycolide acid) (PLGA), poly(l-lactic acid) (PLLA), PLLA*-PLGA copolymer systems, poly(glycerol-sebacate) (PGS), polydimethylsiloxane (PDMS), polydimethylsiloxane (PDMS), poly(methyl methacrylate) (PMMA), poly(ethylene glycol) diacrylate (PEGDA), parylene-C and polycaprolactone (PCL), and any combinations thereof.

6. The graft of claim 1, wherein the thickness of the rehydrated crosslinked scaffold film is between 5 and 10 microns.

7. The graft of claim 1, wherein the retinal pigment epithelium cells are differentiated human embryonic stem cells.

8. The graft of claim 1, wherein the RPE cells are at least one selected from a group consisting of positioned on top of the retinal implant scaffold, integrated within the scaffold.

9. The graft of claim 1, wherein the RPE cells are at least one selected from a group consisting of arranged in a monolayer, positioned separately within a scaffold and any combination thereof.

10. The graft of claim 1, wherein the RPE cells are either poured or printed on said scaffold layer.

11. The graft of claim 1, wherein said scaffold layer is air dried.

12. The graft of claim 11, wherein said air drying is done by adding a predefined volume of said scaffold layer to a mold, setting the desired environmental conditions to predetermined environmental conditions, and letting the solution dry for a predefined period of time.

13. The graft of claim 12, wherein said environmental conditions are selected from a group consisting of humidity, temperature, gas composition, gas flow rate and direction, light radiation and any combination thereof.

14. The graft of claim 1 wherein the graft diameter is 1-8 mm.

15. The graft of claim 1, wherein said cross crosslinking is performed by a cross linking agent being selected from a group consisting of N-hydroxysuccinimide, NHS, 1-ethyl-3-(3-dimethylaminopropyl)carbodiimide hydrochloride), EDC, NHS-EDC solution and any combination thereof.

16. The graft of claim 1, wherein said cross crosslinking is performed at a temperature range of 0-40 degrees Celsius and for a period of time of 1 minute to 24 hours.

17. The graft of claim 1, wherein said cross crosslinking is performed by enzymes.

18. The graft of claim 17, wherein said enzymes is transglutaminase.

19. The graft of claim 1, wherein said cross crosslinking is performed by at least one selected from a group consisting of applying UV light, elevated temperature (35-90 degrees Celsius); or by chemical agents as glutaraldehyde and any combination thereof.

20. A method of manufacturing a retinal graft, said method comprising:
preparing a scaffold material solution;
air-drying the scaffold material solution, to form a thin scaffold layer;
crosslinking the thin scaffold layer;
rehydrating the scaffold layer; and
applying retinal pigment epithelium cells onto the scaffold layer.

21. The method of claim 20, comprising maturing the retinal pigment epithelium cells for at least one week prior to implantation.

22. The method of claim 20, comprising maturing the retinal pigment epithelium cells for at least three weeks prior to implantation.

23. The method of claim 20, wherein preparing a scaffold material solution comprises using a collagen or collagen methacrylate.

24. The method of claim 23, including preparing the collagen or collagen methacrylate from human recombinant collagen.

25. The method of claim 23, including sourcing the collagen or collagen methacrylate from human collagen.

26. The method of claim 20, wherein the crosslinking step comprises introducing EDC and NHS molecules to the scaffold material solution.

27. The method of claim 20, wherein the crosslinking step comprises introducing lithium phenyl-2,4,6-trimethylbenzoylphosphinate (LAP) or 1-[4-(2-Hydroxyethoxy)-phenyl]-2-hydroxy-2-methyl-1-propane-1-one molecules to the scaffold material solution and applying light on it.

28. The method of claim 20, wherein the drying step is performed for 12 hours-7 days.

29. The method of claim 20, wherein the drying step comprises drying in a mold.

30. The method of claim 20, wherein applying retinal pigment epithelium cells onto the scaffold layer is done by seeding, printing, or a combination thereof.

31. The method of claim 30, wherein the printing comprises using laser-induced-forward-transfer.

32. The method of claim 30, wherein the printing comprises using a micro-pipetting or an inkjet.

33. The method of claim 20, further comprising cutting the retinal graft to a predetermined size.

34. The method of claim 20, further comprising cryo-preserving the graft and thawing it prior to implantation.

35. The method of claim 34, wherein the cryo-preserving comprises incubating after the thawing step.
